(19) United States
(12) Patent Application Publication
(10) Pub. No.: US 2021/0283280 A1
(43) Pub. Date: Sep. 16, 2021

(54) COMPLEX POLYSACCHARIDE-BOUND RADIOISOTOPE CHELATES AND METHODS OF TREATING MALIGNANCIES THEREWITH

(71) Applicant: Robert Norman TAUB, New York, NY (US)

(72) Inventor: Robert Norman TAUB, New York, NY (US)

(21) Appl. No.: 16/815,759

(22) Filed: Mar. 11, 2020

(51) Int. Cl. A61K 51/04; A61K 51/02; A61P 35/00

(52) U.S. Cl. CPC A61K 51/0491; A61P 35/00; A61K 51/025; A61K 51/0482

(57) ABSTRACT

The invention provides a compound having the following structure:

D-DT-R,

wherein D is a dextran molecule or a charged dextran molecule having a molecular weight between about 50,000 and about 110,000 Daltons, DT is dodecane tetra-acetic acid (DOTA) or a conjugate base thereof, and R is a radioactive isotope. The invention also provides a method for treating body cavity cancer in a patient afflicted therewith, comprising administering an effective amount of a dextran-dodecane tetraacetic acid-radioactive isotope compound in a pharmaceutically effective vehicle.

### FIELD OF THE INVENTION

The present invention relates to isotope delivery systems and more particularly to complex polysaccharide-bound chelates of radioisotopes, and methods for treatment of malignancies using such chelates.

### BACKGROUND OF THE INVENTION

Irradiation of localized cancer has long been known as an effective means of treatment that must be engineered to avoid damaging adjacent healthy tissue. Use of radiation beam therapy can be effective in killing cancer cells, but also can cause damage to adjacent organs and tissue. Likewise, intravenous chemotherapy agents affect healthy tissue as well as diseased tissue, and consequently can cause side effects including nausea, vomiting, dizziness, hair loss, and damage to healthy organs including the liver and kidneys, among others. As a result, various attempts have been made to develop localized cancer treatments to avoid unnecessary irradiation of healthy tissue or exposure to harsh chemotherapy agents. This is especially true for superficial non-muscle invasive bladder tumors, synovial exuberant tissue, and disseminated ovary, appendix or peritoneal cancers confined to the abdominal cavity. These tumors are adjacent to vital, highly radiosensitive organs such as the kidneys and the intestines. It is difficult to avoid damage to healthy tissues from radiation applied from an external source that travels in a straight line to an irregular cavitary target. Many previous attempts included intravenously injected radioactivity that passed through the entire body, a small portion of which eventually was attracted to the intended target by specific antibodies. Other approaches involved relatively low molecular weight radioactive salts, which quickly passed out of the injected cavity into the bloodstream from which they disseminated widely.

Other approaches involved localized administration of chemotherapy agents coupled to a biocompatible matrix to form a treatment solution,_but were limited by the barrier to diffusion of the drug posed by the poorly permeable bladder mucosa. For example, U.S. Pat. No. 9,884,028 (Holzer); U.S. Pat. No. 10,471,150 (Konorty); and European Patent Specification No. EP 525 777 B1 (Holzer) discuss such an approach for coating an internal cavity with a treatment solution. The treatment solution can include a solidifiable matrix that is coated on the interior of an internal cavity, and acts a slow release delivery system for such common chemotherapy agents as Taxol, doxorubicin, mitomycin C. The foregoing patents and applications, as well as the cited references below, are incorporated by reference herein with the same force and effect as set forth herein. The present invention seeks to remedy the deficiencies of previous methods used to treat cancer and other diseases in internal cavities, while protecting unaffected areas.

### SUMMARY OF THE INVENTION

The present invention provides a compound having the following structure:

D-DT-R,

wherein D is a dextran molecule or a charged dextran molecule having a molecular weight between about 50,000 and about 110,000 Daltons, DT is dodecane tetra-acetic acid (DOTA) or a conjugate base thereof, and R is a radioactive isotope. The radioactive isotope may be yttrium-90, technetium-199, Indium-111, gadolinium-86, actinium-225, bismuth 213, lutetium-177 inter alia. In one embodiment, D is dextran-70 and R is yttrium-90, indium 111, or technetium-199. In another embodiment, the dextran molecule includes between about 150,000 and about 400,000 glucose subunits. In a further embodiment the invention comprises an effective cancer therapeutic amount of the D-DT-R in a pharmaceutically acceptable vehicle. In yet another embodiment, the dextran is Dextran 70 and the radioactive isotope is ytterium-90, technetium-199, gadolinium-86, actinium-225, Bismuth 213, indium 111 or lutetium-177. In an additional embodiment, the cancer is found in a body cavity and the cancer in the body cavity is bladder cancer, peritoneal cancer, appendiceal carcinoma, ovarian carcinoma, abdominal cancer of unknown primary, pleural mesothelioma, or metastatic breast or lung cancer involving the pleural cavity.

In another embodiment of the invention, the invention provides a method for treating body cavity cancer in a patient afflicted therewith, comprising administering an effective amount of a dextran-dodecane tetraacetic acid-radioactive isotope compound in a pharmaceutically effective vehicle. The dextran may have a molecular weight between about 50,000 and about 110,000 Daltons and the radioactive isotope is yttrium-90, technetium-199, gadolinium-86, actinium-225, lutetium-177, indium. The cancer in the body cavity is bladder cancer, peritoneal cancer, appendiceal carcinoma, ovarian carcinoma or pleural primary or metastatic cancer. In one embodiment, the effective amount comprises administering between about 25 and 75 mCi of radiation to an affected region.

### BRIEF DESCRIPTION OF THE DRAWINGS

The invention can be better understood by reviewing the following detailed description of the preferred embodiments with reference to the drawings, in which:

FIG. 1 is structural drawing of dextran;

FIG. 2 is a three-dimensional structural drawing of dextran; and

FIG. 3 is a structural drawing of DOTA.

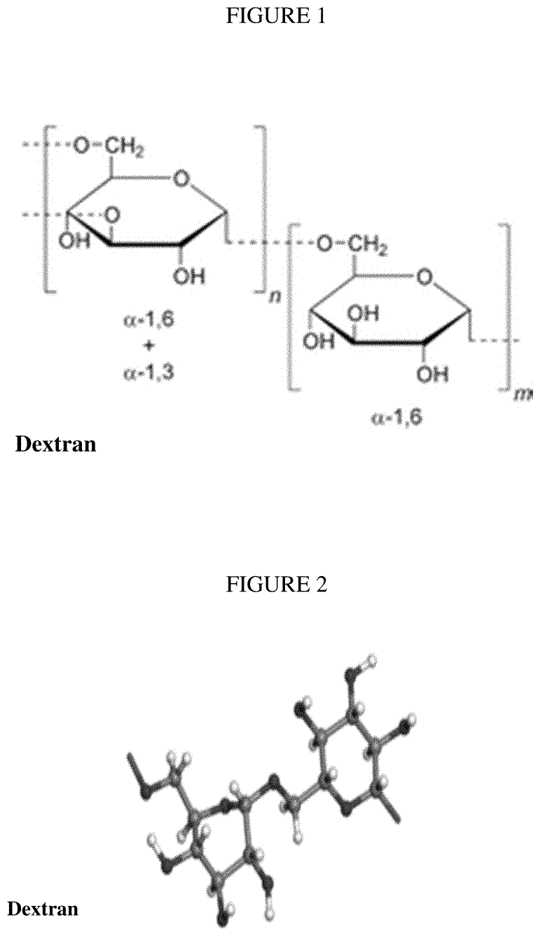

### DETAILED DESCRIPTION OF THE PREFERRED EMBODIMENTS

In the proposed method, Dextran 70 (see figure), which is generally recognized as safe, biologically inert, and membrane-impermeable, is covalently bound to a DOTA (see FIG. 2) chelated radioisotope as a conformable radiation source that can be introduced into and optionally withdrawn from a diseased cavity such as a cancerous urinary bladder or abdomen. Because of its large molecular size and inability to penetrate cavity membranes, the dosage of administered radioactivity will be limited and defined by the contours, regular or irregular of the body cavity into which it is injected, and the time during which it is permitted to remain. Normal organs will be largely spared.

As used herein, the term “internal cavity” or “body cavity” refers specifically to certain spaces or potential spaces that surround or protect organs that are lined by specialized epithelial cells that are moist or contain liquid. Examples are the pleural and peritoneal/pelvic spaces, and synovial spaces surrounding joints. The term “bladder” used herein refers specifically to the urinary bladder. The definition also includes artificially made or enlarged cavities in adipose tissues and fibrous capsules in internal organs such as the kidney, heart, intestine, etc. that are accessible by image guided laparoscopic techniques.

DEXTRAN—Of the many polymers available, Dextran 70, a branched-glucose polymer has many binding sites, a high-molecular weight, is commercially abundant, and is acknowledged to be virtually non-toxic. Dextran 70 is extensively used as a plasma expander to sustain victims of wartime or surgical bloodshed, in which case it circulates comfortably within the vascular system with minimal side effects. Dextran has no medicinal properties, and is not itself intended to treat any human disease. In the proposed use, Dextran 70 would serve as an inert molecular scaffold for a therapeutic radioisotope, imparting a large enough molecular weight to the conjugate to be impermeable to many physiological membranes, including those that bound the vascular system.

Each Dextran 70 molecule has approximately 200,000 glucose units, and thus can accommodate many DOTA molecules. Twenty molecules of DOTA would not alter the stability of the molecule, which can chelate high-specific activity Yttrium-90, and inter alia, Lutetium-177, and Technetium-199, Gadolinium-86, or even the alpha-emitter Actinium 212. Dextran, like many branched polysaccharides, has many reactive hydroxyl groups at which chemical bonds can be constructed. It is available as a purified substance in many molecular weight forms from 1000 to 2,000,000. Dextran 70 has approximately 200,000 of such glucose monomers or determinants. It is possible that using a final molecular weight of 70 kilodaltons would allow selective diffusion into tumors which have a greater permeability than normal tissue (see L. W. Seymour, “Critical Review of Therapeutic Drug Support Systems,” vol. 9, pages 135 to 187 (1991)). Higher molecular weights might be used for the bladder where only the penetration of the crevices would be important. Much pharmacological data for Dextran are available based on its wide use as a plasma volume dilator, where it has been shown to persist for several weeks after infusion in patients and during which time it is gradually oxidized into smaller forms and eventually removed by the kidneys. With judicious use of radioactive materials with short half-lives, the deterioration of the drug should parallel a reduction in the beta radiation emitted (See Goodman and Gilman, Pharmacological Basis of Therapeutics (8th ed.), pages 690-91).

DEAE-Dextran: In addition to the uses proposed above, there may be benefit to the use of Dextran carrying positive charges such as those conferred by addition of Diethylaminoethyl dextran, which would facilitate electrostatic binding between DEAE-Dextran and normal and preferentially neoplastic transitional epithelium of the bladder mucosa. DEAE dextrans of 500000 MW may be used as Active Pharmaceutical Ingredients (API) or in pharmaceutical preparations e.g. as a vaccine adjuvant, a transfection agent for gene therapy, and as an ingredient in cholesterol lowering products. When tested for its ability to adhere to bladder mucosa. “The result clearly demonstrated a charge-dependent difference in the quotient of radioactive uptake in tumor tissue: normal tissue. Instillations of cationic dextran yielded a high quotient, up to 3000. Normal tissue had background activity. Anionic dextran yielded a low quotient, 1.8-2, with increased background (i.e. uptake in normal tissue). Neutral dextran gave a quotient of up to 90. No radioactivity could be detected in blood.” Holmberg, A. R., Wilchek, M., Marquez, M., Westlin, J. E., Du, J., Nilsson, S., “Ion Exchange Tumor Targeting: A New Approach,” Clin. Cancer Res. 1999; 5 (Suppl): 3056s-Es. Gram amounts of DEAE-dextran may be taken by mouth with no ill effect; however the molecule is known to be an anti-heparin, and could conceivably interfere with normal hemostasis. Its judicious use for treatment of bladder cancer may nevertheless be warranted, and is therefore included as a substance appropriate for conjugation.

DOTA—Dodecane tetraacetic acid, short for 1,4,7,10-Tetraazacyclododecane-1,4,7,10-tetraacetic acid, also called Tetraxetan is shorthand for both the tetracarboxylic acid and its various conjugate bases. The four secondary amine groups are modified by replacement of the N—H centers with N—CH₂CO₂H groups. The resulting aminopolycarboxylic acid, upon ionization of the carboxylic acid groups, is a high affinity chelating agent for divalent and trivalent cations. As a polydentate ligand, DOTA envelops metal cations, especially the lanthanides such as Yttrium and in such complexes DOTA functions as an octadentate ligand, binding the metal through four amine and four carboxylate groups. Most such complexes feature an additional water ligand, giving an overall coordination number of nine. DOTA can be conjugated to the glucose residues of Dextran by attachment of one of the four carboxyl groups as an amide, although other configurations are possible. The remaining three carboxylate anions are available for binding to the Yttrium ion.

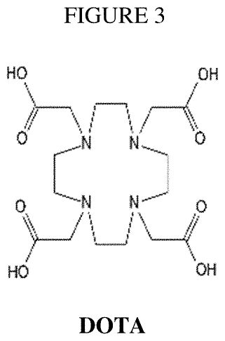

CONJUGATION—formation of DOTA-Dextran70 (Dextran-Y®): Earlier lengthy and laborious techniques of conjugation involved procedures for binding of Dextran to DOTA. This procedure has undergone much change over the past two decades. Earlier methods involved activation of the hydroxyl units of Dextran with allyl bromide (Gedda, L. I., Olsson, P., Ponten, J., Carlsson, J., Development and In Vitro Studies of Epidermal Growth Factor-Dextran Conjugates for Boron Neutron Capture Therapy,” Bioconjug. Chem. 1996 September-October; 7(5):584-91.), followed by reaction with cysteamine at 50° C. and pH 11 (maintained by dropwise addition of 2.5 N NaOH) for 3 hours, neutralization with acetic acid, dialyzed against deionized water, concentrated, and the product, allyl dextran, is stored lyophilized at −80° C. The average molecular diameter is measured by diffusion with laser light (Honeywell MicroTrac UPA 150). In the second step, the allyl groups react with aminoethanethiol (cysteamine) in DMSO (30 ml) to produce ligands with amino terminals. This reaction is started with ammonium persulfate (99.99%, 1.0 g) and is carried out under a nitrogen atmosphere for 3 hours; reaction volume is doubled with deionized water and the solution is adjusted to pH 4 with sodium hydroxide (2.5 N), and acetate buffer, ultra-filtered (5 mm), dialyzed with acetate buffer and deionized exchange water, lyophilized and stored at ˜80° C. Finally, the mixed anhydride method (Krejcarek, G. E., Tucker, K. L., “Covalent Attachment of Chelating Groups to Macromolecules,” Biochem. and Biophys. Res. Comm. 77(2) 581-585, 1977.) was used to conjugate to a similar chelator, DTPA, carrying the lanthanide to the dextran structure. The synthesis begins with activation of the chelator (20 g) with Isobutyl Chloroformate (IBCF) (3.1 ml) in acetonitrile (83 ml) at −30° C. which is then added slowly to dextran with amino terminals (2 g) in bicarbonate buffer (0.1 M, pH 9) at 4° C. (see FIG. 3) and stirred overnight, and dialyzed as above, lyophilized and stored at −80° C.

More recently the synthesis has been facilitated by the availability of (P-SCN-BN-DOTA (Chemical Formula: C₂₄H₃₃N₅O₈S.2.5HCl-2.5H₂O: Chemical Name: S-2-(4-Isothiocyanatobenzyl)-1,4,7,10-tetraazacyclododecane tetraacetic acid). This bifunctional reagent can directly couple to amino-functionalized Dextrans.

Thus, it can be understood by a person of skill in the art that the present invention provides several closely related methods to improve upon current radiotherapy of body cavities by providing the novel alternative of a liquid radioisotope injected into and confined within a cavity in which it can irradiate surfaces involved by tumor.

Specifically, the invention employs a Dextran polymer of about 70,000 Daltons molecular weight, a versatile biologically compatible material that is covalently bound to a metal chelating agent, DOTA, to localize Yttrium 90, or other radioactive isotopes in close proximity to inner surfaces of body cavities so as to impart in one embodiment relatively uniform radiation to inner layers of the bladder afflicted with superficial non-muscle invasive bladder cancer. In another embodiment, the invention provides a method to treat superficial layers of peritoneum afflicted by widespread military or surface tumors arising in the distal fallopian tube of the ovary. In yet another embodiment, the invention provides a method of treating cancer in cases of peritoneal involvement by various other tumors knows to be relatively confined to the peritoneal cavity for long periods before dissemination through the body, such as inter alia, appendiceal carcinoma, primary peritoneal carcinoma, peritoneal mesothelioma, among others. In another embodiment the present invention provides a method irradiating the synovial cavity of patients suffering from advanced proliferative synovial secretory inflammation. In a further embodiment, the radioactive fluid can be embedded in sepharose granules or thixotropic gels in difficult—to resect or irradiate abdominal recesses, such as the area surrounding the superior mesenteric artery after Whipple resection.

In these embodiments, the dextran-70-DOTA-Yttrium conjugate, (hereby abbreviated “Dextrad-Y”) would be tested in humans in a Phase 0 model, i.e., using surrogate isotopes such as Technetium or Indium in clinically minute, deeply sub-therapeutic amounts, during valid surgical or other procedures scheduled in these patients, after obtaining informed consent as volunteers for additional maneuvers to determine proof of principle, and possibility of efficacy, of a new treatment so as to pave the way for a formal clinical trial.

In the first embodiment, 200-300 ml solution of Dextrad-Y will be introduced into the bladder of a fluid-deprived subject just after voiding via Foley Catheter, which is sealed off and allowed to remain for 4-6 hours, after which it would be flushed until less than 1% of injected radioactivity is judged to remain. If available, cystoscopic biopsies of normal and cancerous bladder tissue will be removed and sampled for radioactivity and histologic change. Samples of blood and urine will be tested for leakage of radioactivity into the bloodstream. This will pave the way for a formal Phase I testing of higher Yttrium doses in the range of 25 to 150 mCi.

In the second embodiment, patients with recently surgically debulked Ovarian Carcinoma will undergo placement of two intraperitoneal ports, for instillation of postoperative chemotherapy. At that time, usually at 2-3 weeks postoperatively, sub-therapeutic doses of Dextrad-Technetium will be instilled into the abdomen in 1-2 liters of saline or similar isosmotic fluid, and monitored for uniformity of dispersal and leakage of radioactivity into the bloodstream. At 4-6 weeks post injection, the fluid will be sampled to determine if a reasonable gradient of >10:1 exists between the peritoneal cavity and the blood, paving the way for a similar “second look” maneuver at six months, this time with high-dose Dextrad-Y (up to 100 mCi) Yttrium 90 given with full shielding, and appropriate radiation precautions.

In the third embodiment, patients with disseminated neoplasm confined to the abdominal cavity that have undergone debulking surgery and have had ports placed for intraperitoneal chemotherapy, will, at three weeks undergo radiological study with dilute barium to determine distribution of injected material, after which they will be given sub-therapeutic doses of Dextrad-Technetium and reimaged. If the distribution is acceptably thorough up to 100 mCi, Dextrad-Y will then be given as therapy.

In the fourth embodiment, synovial spaces of patient with refractory synovial inflammatory proliferation would be injected with sub-therapeutic doses of Dexrad-Technium, and if the distribution is adequate, and systemic and local leakage is minimal, three days later the patient will receive a single injection of 10-15 mCi of Dextrad-Y, sufficient to sclerose the joint space.

In the fifth embodiment, Dextrad-Y will be incorporated into sepharose granules designed to retard molecules of less than 2 million molecular weight, and the granules will then be mixed with a preparation of surgical fibrinogen/gelatin (e.g., Gelfoam®). This material would be used specifically to assist the radiation of the surgical fossa created around the superior mesenteric artery during the Whipple Pancreatectomy procedure to effect additional local control, previously only achievable with great difficulty with brachytherapy (no longer used).

Thus, the present invention includes the novel method and procedure for using a Dextran structure to modify and shape the field of administration of radiotherapy of cavities and associated structures. The method of its use, namely to inject or instill the assembled radioactive construct as a large molecular weight inert construct which will remain largely confined to the cavity in which it is injected, including the bladder, peritoneal cavity, synovial cavity, and in gel form into secondary spaces such as the lesser sac and origin of the superior mesenteric artery. This formulation does not exclude the use of Dextrans of other molecular weights conjugated with DOTA, which can form increasingly viscous or gel like fluids or can be admixed with other materials such as Gelfoam® to conform appropriately to different spaces for appropriate radiotherapy.

Although the invention has been described in conjunction with specific embodiments thereof, it should be apparent to one of skill in the art that many alternatives, modifications, and variations will be apparent upon review of this disclosure. It is therefore intended to embrace all such alternatives, modifications, and variations that fall within the spirit and scope of the invention as defined by the following claims.

1-15. (canceled)

16. A compound having the following structure:
D-DOTA-R,
wherein D is a diethylaminoethyl dextran having a molecular weight between about 50,000 and about 110,000 Daltons, DOTA is dodecane tetra-acetic acid or a conjugate base thereof, and R is a radioactive isotope.

17. The compound of claim 16, wherein the radioactive isotope is yttrium-90, technetium-99(m), gadolinium-68, actinium-225, lutetium-177, indium, ytterbium, radium, cesium, or iridium.

18. The compound of claim 16, wherein the diethylaminoethyl dextran has a molecular weight of about 70,000 Daltons.

19. A composition for treating a body cavity cancer in a patient afflicted therewith, the composition comprising a therapeutically effective amount of the compound of claim 16, wherein the compound is confined within the body cavity and is unable to penetrate membranes in the body cavity.

20. The composition of claim 19, wherein the composition further comprises a pharmaceutically acceptable vehicle.

21. The composition according to claim 19, wherein the cancer is bladder cancer, peritoneal cancer, appendiceal carcinoma, or ovarian carcinoma.

22. A method for treating a body cavity cancer in a patient afflicted therewith, comprising introducing into the body cavity of the patient a therapeutically effective amount of a compound with a formula of D-DOTA-R, wherein D is a diethylaminoethyl dextran having a molecular weight between about 50,000 and about 110,000 Daltons, DOTA is dodecane tetra-acetic acid or a conjugate base thereof, and R is a radioactive isotope, wherein the compound is confined within the body cavity and unable to penetrate membranes in the body cavity.

23. The method of claim 22, wherein the radioactive isotope is yttrium-90, technetium-99(m), gadolinium-68, actinium-225, lutetium-177, indium, ytterbium, radium, cesium, or iridium.

24. The method of claim 22, wherein the radioactive isotope is yttrium-90 or bismuth-213.

25. The method of claim 22, where the body cavity cancer is bladder cancer, peritoneal cancer, appendiceal carcinoma, or ovarian carcinoma.

26. The method of claim 22, wherein the therapeutically effective amount of the compound is between about 25 and 75 mCi and the radiation is substantially confined to the body cavity.

27. The method of claim 22, wherein the compound is introduced into the cavity with a pharmaceutically acceptable vehicle.

28. The method of claim 22 further comprising withdrawing the compound from the body cavity after a predetermined treatment period.

29. The method of claim 28, wherein the body cavity is flushed with an eluent until the body cavity is essentially free of the compound.

30. The method of claim 29, wherein the body cavity is flushed with the eluent until less than 1% radioactivity from the compound remains.

31. The method of claim 28, wherein the treatment period is about 4-6 hours.

32. A method for treating a body cavity cancer in a patient afflicted therewith, comprising introducing into the body cavity a therapeutically effective amount of a compound with a formula of D-DOTA-R, wherein the compound is confined within the body cavity, wherein D is a positively charged dextran having a molecular weight between about 50,000 and about 110,000 Daltons, DOTA is dodecane tetra-acetic acid or a conjugate base thereof, and R is a radioactive isotope, wherein the compound is unable to penetrate membranes in the body cavity and wherein the radiation is substantially confined to the body cavity.

33. The method of claim 32, wherein the radioactive isotope is yttrium-90, technetium-99(m), or bismuth 213.

34. The method of claim 32, wherein the radioactive isotope is yttrium-90, technetium-99(m), gadolinium-68, actinium-225, lutetium-177, indium, ytterbium, radium, cesium, or iridium.

35. The method of claim 32, wherein the therapeutically effective amount of the compound is between about 25 and 75 mCi.
